(19) United States
(12) Patent Application Publication
(10) Pub. No.: US 2013/0041020 A1
(43) Pub. Date: Feb. 14, 2013

(54) Methods of Control and Prophylaxis of Inflammation and Mitigation of Inflammatory Conditions in Companion Animals

(71) Applicant: Nolan Zebulon Frantz, Andover, NJ (US)

(72) Inventor: Nolan Zebulon Frantz, Andover, NJ (US)

(73) Assignee: Hill's Pet Nutrition, Inc., Topeka, KS (US)

(21) Appl. No.: 13/642,561

(22) PCT Filed: May 9, 2011
(86) PCT No.: PCT/US11/35736

§ 371 Date: Oct. 22, 2012

Related U.S. Application Data
(60) Provisional application No. 61/334,037, filed on May 12, 2010.

(51) Int. Cl. A61K 31/385; A61P 19/02; A61P 29/00

(52) U.S. Cl. 514/440

(57) ABSTRACT

The invention relates to methods of control and prophylaxis of inflammation and mitigation of inflammatory conditions, particularly arthritis and joint pain, in companion animals, e.g., dogs or cats, comprising administering a diet comprising lipoic acid.

### FIELD OF THE INVENTION

The invention relates to methods of control and prophylaxis of inflammation and mitigation of inflammatory conditions, particularly arthritis and joint pain, in companion animals, e.g., dogs or cats, comprising administering a diet comprising lipoic acid.

### BACKGROUND OF THE INVENTION

Lipoic acid (LA) is an organosulfur compound derived from octanoic acid. LA contains two vicinal sulfur atoms (at C6 and C8) attached via a disulfide bond and is thus considered to be oxidized (although either sulfur atom can exist in higher oxidation states). The carbon atom at C6 is chiral and the molecule exists as two enantiomers R-(+)-lipoic acid (RLA) and S-(−)-lipoic acid (SLA) and as a racemic mixture R/S-lipoic acid (R/S-LA). Only the R-(+)-enantiomer exists in nature and is an essential cofactor of four mitochondrial enzyme complexes. Both RLA and R/S-LA are available as over-the-counter nutritional supplements and have been used nutritionally and clinically since the 1950s for a number of diseases and conditions. LA has been used in animal food, for example Hill's Prescription Diet canine b/d®, which is believed to enhance alertness and cognition particularly in older dogs.

Osteoarthritis is a chronic, degenerative joint disease that is caused by the progressive inflammation and deterioration of the cartilage, bone, and soft tissue of one or more joints. Rheumatoid arthritis is an autoimmune condition that causes inflammation and damage to the joints. Both are chronic inflammatory conditions and are common in older dogs and cats. Because the damage to the joints is progressive and largely irreversible, it is desirable to identify and address the inflammatory process proactively.

### SUMMARY OF THE INVENTION

The invention provides methods of control and prophylaxis of inflammation and mitigation of inflammatory conditions, particularly arthritis and joint pain, in companion animals, e.g., dogs or cats, comprising administering a diet comprising lipoic acid, e.g., for a period of at least two weeks, e.g., wherein the diet comprises a food having 10-10,000 ppm of lipoic acid, e.g., a dry food comprising 50-200 ppm lipoic acid.

In a further embodiment, the invention provides methods of control and prophylaxis of inflammation and mitigation of inflammatory conditions, particularly arthritis and joint pain, in companion animals, e.g., dogs or cats, comprising measuring expression of biomarkers for inflammation, and administering a diet containing lipoic acid, e.g., for a period of at least two weeks, e.g., wherein the diet comprises a food having 10-10,000 ppm of lipoic acid, e.g., a dry food comprising 50-200 ppm lipoic acid, to reduce expression of one or more of the biomarkers for inflammation.

### DETAILED DESCRIPTION OF THE INVENTION

The diet for use in the methods herein includes for example, a canine diet comprising at least 500 IU/kg Vitamin E, e.g., 500-2000 IU/kg, at least about 40 ppm vitamin C, e.g., 40-200 ppm, at least 50 ppm carnitine, e.g., 50-300 ppm and at least 50 ppm lipoic acid, for example 50-250 ppm, for example a canine diet comprising:

| Nutrients, | |
|---|---|
| 100% Dry Matter Basis | Amount |
| Crude Protein, % | 15-25 |
| Fat, % | 10-20 |
| Crude Fiber, % | 2.5-7.5 |
| Total dietary fiber, % | 10-25 |
| Soluble fiber, % | 0.5-3 |
| Calcium, % | 0.1-1 |
| Phosphorus, % | 0.1-1 |
| Vitamin E, IU/kg | 500-2000 |
| Vitamin C, ppm | 40-200 |
| Carnitine, ppm | 50-300 |
| Lipoic acid, ppm | 50-250 |
| Vegetable blend, % | 1-10 |

The lipoic acid is, for example, R-(+)-lipoic acid (RLA) and S-(−)-lipoic acid (SLA) or a racemic mixture R/S-lipoic acid (R/S-LA), preferably RLA or R/S-LA, in free or nutritionally acceptable salt or ester form, preferably in free form or sodium salt form.

The biomarkers for inflammation include, for example, any one or more of the following: Tumor necrosis factor alpha, GM-colony stimulating factor, Monocyte chemotatic protein-1, Interferon gamma, Interleukin-10, Interleukin-15, Interleukin-18, Interleukin-2, Interleukin-4, Interleukin-6, Interleukin-7, Interleukin-8, Interferon gamma induced protein-10, KC chemokine.

### EXAMPLE 1

### Effect of Lipoic Acid Diet on Inflammatory Biomarkers in Geriatric Dogs

A study was conducted to evaluate the effect of a food composition on inflammatory biomarkers and gene expression in healthy geriatric dogs. Twenty-nine geriatric beagle dogs (initial weight, 13.51±1.66 kg, age, 10.7±2.33 years) were included in the study. All dogs were fed a control maintenance food for 28 days followed by the test food. The test food contains increased levels of omega 3 fatty acids, lipoic acid, antioxidants from a fruit and vegetable blend, Vitamins C and E, and L-carnitine. Serum and whole blood samples were collected on the last day of the control food (day 0) and after 14 days on the test food. Inflammatory and hormone biomarkers as well as gene expression changes were measured. RNA was extracted according to the procedures provided in the PAXgene Blood RNA Kit Handbook (Qiagen, Valencia, Calif.). RNA was hybridized to an Affymetrix GeneChip Canine-2 Genome Arrays and normalized using Robust Multi-Array Average. Transcripts having a P<0.05 (following a false discovery rate adjustment value of 0.1) and a fold change range of at least 1.3 were considered different. After consuming the test food, geriatric dogs fed the test food had lower IL-4, IL-6, IL-10, KC, and total cytokines. No differences in hormone biomarkers were detected. Geriatric dogs fed the test food also had 1123 genes that were up or down regulated compared to day 0. Of these, genes associated with amyloid beta plaque formation were down-regulated while genes associated with clearance of beta amyloid plaque were up-regulated. In addition, genes associated with neurotransmitter signaling and cell adhesion were significantly up-regulated when geriatric dogs were fed the test food compared to the day 0 on the control. This study provides gene expression evidence that support the benefits observed in geriatric dogs for memory and behavior associated responses previously shown with the test food.

The test food has been shown to improve memory and cognition in geriatric dogs to a level similar to young dogs as measured by task and discrimination testing. However, the underlying mechanism by which these improvements are observed has not been defined. Inflammation plays a major role in development of many disease processes including general aging. In addition, biochemical measures related to cognitive improvement such as gene expression have only seen limited use in the dog.

The objective of this study was to determine the effect of the test food on inflammatory and hormone biomarkers as well as gene expression changes in geriatric dogs.

Twenty-nine neutered/spayed beagle dogs were identified for this study and fed the test food for 28 days. Dogs were considered healthy by physical exam and serum chemistry. The study protocol was reviewed and approved by the Institutional Animal Care and Use Committee. All dogs were immunized against canine distemper, adenovirus, parvovirus, bordetella, and rabies, and none had chronic systemic disease on the basis of results of physical examination, complete blood count determination, serum biochemical analyses, urinalysis, and fecal examination for parasites. Dogs experienced behavioral enrichment through interactions with each other, by daily interaction and play time with caretakers, daily opportunities to run and exercise outside and access to toys. Prior to sample collection at day 0, all dogs were fed a basal maintenance control food for 28 days. Dogs were then switched to the test food and samples collected after 14 days. Blood was drawn and collected into PAXgene tubes and stored at −80° C. until evaluation. Canine-2 Affymetrix Genechip microarrays were used to identify gene differences at day 14 compared to day 0.

Plasma biomarkers—A paired t-test analysis was used to determine changes in inflammatory and hormone biomarkers using SAS version 9.0 in response to food. Total cytokines were calculated using all cytokines.

Genes—Gene expression was normalized using Robust Multi-Array average and Partek analysis tool was used to determine differences. Genes having a P<0.05 (following a false discovery rate adjustment of Q=0.1) and a fold-change of at least 1.25 were considered different among the two groups. Up-regulated genes are shown as positive fold-changes. Down-regulated genes are shown as negative fold-changes.

TABLE 1
Contents of the Diets

| Nutrients, | | |
|---|---|---|
| 100% Dry Matter Basis | Canine control food | Canine test food |
| Crude Protein, % | 18.45 | 20.36 |
| Fat, % | 14.48 | 15.52 |
| Crude Fiber, % | 2.05 | 4.34 |
| Total dietary fiber, % | 7.22 | 13.23 |
| Soluble fiber, % | 0.65 | 1.63 |
| Calcium, % | 0.66 | 0.67 |
| Phosphorus, % | 0.60 | 0.58 |
| Vitamin E, IU/kg | 94 | 1183 |
| Carnitine, ppm | 10 | 291 |
| Lipoic acid, ppm | 0 | 101 |
| Vegetable blend, % | 0 | 6.30 |

TABLE 2
Inflammatory and hormone biomarkers measured in plasma in
response to test food (SE = Standard error of the mean):

| | | | | Paired |
|---|---|---|---|---|
| | | | | t-test |
| | Day 0 | Day 14 | | day 0 vs |
| Plasma Biomarker | Mean | Mean | SE* | day 14 |
| Inflammation markers | | | | |
| Tumor necrosis factor | 38.37 | 19.99 | 19.83 | 0.38 |
| alpha, pg/mL | | | | |
| GM-colony stimulating | 135037 | 116079 | 90248 | 0.21 |
| factor, pg/mL | | | | |
| Monocyte chemotatic | 19333 | 5010.5 | 13695 | 0.16 |
| protein-1, pg/mL | | | | |
| Interferon gamma, | 2993.9 | 2008.2 | 2158.2 | 0.12 |
| pg/mL | | | | |
| Interleukin-10, pg/mL | 97.86 | 55.11 | 64.56 | 0.05 |
| Interleukin-15, pg/mL | 1040000 | 697909 | 745624 | 0.32 |
| Interleukin-18, pg/mL | 17506 | 4242.8 | 12107 | 0.21 |
| Interleukin-2, pg/mL | 205257 | 71425 | 144969 | 0.16 |
| Interleukin-4, pg/mL | 11649 | 6353.6 | 6131.4 | 0.06 |
| Interleukin-6, pg/mL | 1387.2 | 793.21 | 709.61 | 0.01 |
| Interleukin-7, pg/mL | 80833 | 14206 | 62203 | 0.24 |
| Interleukin-8, pg/mL | 704.85 | 221.90 | 542.06 | 0.28 |
| Interferon gamma | 2.49 | 2.51 | 1.08 | 0.97 |
| induced protein-10, | | | | |
| pg/mL | | | | |
| KC chemokine, pg/mL | 171.54 | 126.61 | 39.66 | 0.01 |
| TOTAL | 76.83 | 37.20 | 19.4 | 0.05 |
| CYTOKINES, ng/mL | | | | |
| Hormone markers | | | | |
| Insulin, pg/mL | 506.23 | 494.22 | 122.31 | 0.92 |
| Adiponectin, ng/mL | 10.53 | 12.01 | 3.36 | 0.16 |
| Leptin, pg/mL | 286.74 | 212.20 | 270.44 | 0.79 |
| Glucagon, pg/mL | 91.38 | 106.66 | 16.40 | 0.33 |
| Resisten, pg/mL | 10799 | 11838 | 1164.2 | 0.13 |

TABLE 3
Genes associated with brain health and function:

| Gene name | Fold change |
|---|---|
| Related to brain health | |
| Amyloid beta A4 | −1.3 |
| Ataxin 7 | −1.3 |
| Alpha 2 macroglobulin | 1.3 |
| Brain derived neurotrophic factor | 1.3 |
| Related to other brain diseases | |
| Ceroid-lipofuscinosis neuronal 5 | −1.5 |
| Related to neural cell adhesion | |
| Contactin 1 | 1.5 |
| Contactin 6 | 1.7 |
| Neuronal cell adhesion molecule | 1.4 |
| Neruonal cell adhesion molecule 2 | 1.5 |
| Neuronal cell adhesion molecule short isoform | 1.4 |
| Related to neurotransmitter signaling and transporters | |
| GABA receptor A | 1.3 |
| GABA receptor B | 1.3 |
| Glutamate receptor 5 | 1.3 |
| Glutamate receptor 6 | 1.3 |
| Glutamate receptor 8 | 1.3 |
| NMDA 2B | 1.4 |
| Neuritin 1 | 1.3 |
| Acetylcholine receptor alpha 6 | 1.3 |
| SLC6A15 | 1.3 |
| SLC6A16 | 1.3 |
| SLC6A18 | 1.3 |
| Cadherin 2 | 1.3 |
| Sodium- and chloride-dependent neurotransmitter | 1.3 |
| transporter NTT5 | |

Clinical studies have shown that feeding the test food to geriatric dogs can help restore youthful energy and improve memory/task ability to a similar level of young adult dogs. In the current study, switching healthy geriatric dogs to the test food after feeding a maintenance control food, demonstrated a decrease in inflammatory markers IL-4, IL-10, IL-6, KC, and total cytokines. Additionally, genes associated with amyloid beta plaque formation were decreased while other genes related to beta-amyloid removal or neurotransmitter signaling were increased.

This study suggests that feeding the test food has a positive effect on quality of life in geriatric dogs through decreasing inflammatory biomarkers and potentially through altering expression of brain health related genes.

1. A method of control or prophylaxis of inflammation and/or mitigation of inflammatory conditions in a dog comprising administering a diet comprising from 50-250 ppm lipoic acid from 500-2000 IU/kg vitamin E, from 40-200 ppm vitamin C, from 50-300 ppm carnitine and from 1-10% on a dry matter basis of vegetable blend.

2. The method of claim 1 wherein the diet is administered for a period of at least two weeks.

3. The method of claim 1, wherein the condition to be controlled, prevented or mitigated is arthritis.

4. The method of claim 1, wherein the condition to be treated is controlled, prevented or mitigated is osteoarthritis.

5. The method of claim 1 wherein the lipoic acid is R-(+)-lipoic acid (RLA) or a racemic mixture R/S-lipoic acid (R/S-LA), in free or nutritionally acceptable salt or ester form.

6. The method of claim 1, further comprising measuring expression of biomarkers for inflammation.

7. The method of claim 6 wherein biomarkers for inflammation comprise one or more biomarkers selected from tumor necrosis factor alpha, GM-colony stimulating factor, Monocyte chemotatic protein-1, Interferon gamma, Interleukin-10, Interleukin-15, Interleukin-18, Interleukin-2, Interleukin-4, Interleukin-6, Interleukin-7, Interleukin-8, Interferon gamma induced protein-10, and KC chemokine.

8. The use of diet as defined in claim 1 in the manufacture of a pet food for use in a method of control or prophylaxis of inflammation and/or mitigation of inflammatory conditions in a dog.

9. The composition as defined in claim 1 for use in a method of control or prophylaxis of inflammation and/or mitigation of inflammatory conditions in a dog.

10. The use in claim 8 further comprising measuring expression of biomarkers for inflammation.

11. The use in claim 10 wherein biomarkers for inflammation comprise one or more biomarkers selected from tumor necrosis factor alpha, GM-colony stimulating factor, Monocyte chemotatic protein-1, Interferon gamma, Interleukin-10, Interleukin-15, Interleukin-18, Interleukin-2, Interleukin-4, Interleukin-6, Interleukin-7, Interleukin-8, Interferon gamma induced protein-10, and KC chemokine.

12. The composition as defined in claim 9 wherein the use further comprises measuring expression of biomarkers for inflammation.

13. The use in claim 12 wherein biomarkers for inflammation comprise one or more biomarkers selected from tumor necrosis factor alpha, GM-colony stimulating factor, Monocyte chemotatic protein-1, Interferon gamma, Interleukin-10, Interleukin-15, Interleukin-18, Interleukin-2, Interleukin-4, Interleukin-6, Interleukin-7, Interleukin-8, Interferon gamma induced protein-10, and KC chemokine.
